(19) United States
(12) Patent Application Publication
(10) Pub. No.: US 2011/0112606 A1
(43) Pub. Date: May 12, 2011

(54) Semiconductor System Integrated With Through Silicon Vias for Nerve Regeneration

(75) Inventors: Alan Gatherer, Richardson, TX (US); Walter H. Schroen, Dallas, TX (US)

(73) Assignee: TEXAS INSTRUMENTS INCORPORATED, Dallas, TX (US)

(21) Appl. No.: 12/616,932

(22) Filed: Nov. 12, 2009

(51) Int. Cl. A61N 1/00

(52) U.S. Cl. 607/50

(57) ABSTRACT

An integrated circuit (IC) chip (100) expanded to nerve fiber (602) growth in the third dimension by through-silicon via-holes (TSV) (131), with an electrically conductive inner sidewall (303) having a roughness (303a) suitable for supporting the growing fiber and conductive connections (210) to the circuitry (101). The TSVs are fabricated parallel to each other and may be arrayed in regular patterns. The chip, provided with a pad (230) for contacting a nerve end and attaching a neuron, acts as a permanent protective sheath for the parallel growing fibers. Nerve fiber growth is stimulated by combining in the chip electrical and magnetic pulses and neurotrophic factors (603); continuous communication with external monitors is provided. The IC provides each TSV with a signal generator, electric and magnetic field generator, power source, potential sensor, and transceiver. The electronic signals may initiate a predetermined action potential in the adjacent nerve fiber end and a sensor is configured for sensing the action potential in the nerve fiber end.

### BACKGROUND

Embodiments of the invention are related in general to the field of semiconductor devices and processes, and more specifically to the structure and fabrication method of systems for enhancing regeneration and growth of nerves.

Nerves that are injured or severed due to trauma or disease can, in some cases, regenerate naturally over a period of time and grow across the injured area to re-innervate a target tissue. The regeneration occurs primarily with nerves of the peripheral nervous system where the breach between nerve ends is no more than a few millimeters. Larger gaps between nerve ends are sometimes repairable by microsurgical procedures to reestablish contact between severed nerve ends. In cases where substantial injury of a peripheral nerve exists, it is sometimes necessary to insert a graft, usually an autograft, to join the servered ends or to reroute a nerve. To facilitate these surgical techniques, investigators have employed a variety of scaffolds and conduits to act as a guide for nerve growth and to promote rejoinder of nerve ends by the body's natural physiological mechanisms. Therapeutic drugs and/or electromagnetic energy is sometimes used to enhance nerve growth in a desired direction.

For the goal of implementing chronic interfaces to the nervous system, the concept of the sieve electrode as such an interface was introduced about 30 years ago. In some approaches, the micromachined sieves used a 20 to 30 μm thick substrate of silicon as support and iridium-lined pores as active sites. However, the multi-directional growth of nerve fibers was cumbersome to control for implantation in the sieve electrodes; growing nerve fibers in parallel was especially difficult. The sheathing of the growing nerve fiber represented an additional challenge. Furthermore, the recording sites, leads and cables caused numerous technical hurdles to achieve reliable interfaces and insulation by dielectrics, and the channeling of nerve fibers through the sieve pores also had proven problematic. A further challenge has been the establishment of intimate contacts between nerve fibers and the recording substrate for producing records with adequate signal-to-noise ratio. In general, the experimental approaches reported in literature often faced technical difficulties of consistent quality, reliable interfaces between different materials, and methods for developing connectors for continuous data acquisition.

Surgical repairs of nerve injuries have generally been disappointing. For instance, structural contacts between nerve ends may be established and yet nervous function is deficient. Nerve fibers may initially show an increase in myelination and diameter of axons, but subsequently fail or cease to regenerate. In the case of injuries of the central nervous system (CNS), repairing and regrowing damaged nerves has been especially challenging. Although a major problem in CNS axonal regeneration is hindrance by neuroglial scarring, studies have shown that CNS axons can regrow in permissible environments. Efforts towards restoration of contact and function in nerves of the CNS have generally involved the use of therapeutic drugs, differentiation of stem cells into nerve cell phenotypes, and application of electromagnetic stimulation. These multi-faceted approaches make restoration efforts expensive. Nevertheless, there is continued strong interest in developing ways to enhance nerve regeneration in individuals suffering from traumatic nerve injury or nerve damage due to disease.

### SUMMARY

Applicants recognized that the implantation of devices for facilitating nerve regrowth requires small yet directionally controlled devices, which offer provisions to stimulate the growing nerve fibers ad lib. by neurotrophic factors and electrical and magnetic pulses. Applicants further saw that the high reliability expected of implanted devices requires a device manufacturing technology, which is fully developed, clean, flexible, and low cost.

Applicants discovered that integrated circuitry, fabricated by standard technology on two-dimensional silicon chips in ultra-clean wafer fabs, can be expanded to nerve fiber growth in the third dimension, when through-silicon via-holes (TSV) are added to the chip. The TSVs are provided with an electrically conductive inner sidewall (preferably using a noble metal) having a roughness suitable for supporting the growing fiber and with conductive connections to the circuitry outside. The TSVs are fabricated parallel to each other and may be arrayed in regular patterns, for example in rows and lines spaced at a predetermined pitch center-to-center. The chip, provided with a pad for contacting a nerve end and attaching a neuron, acts as a permanent protective sheath for the growing fibers.

Having a low cost approach to fabricate a body-implantable device for growing and protecting multiple nerve fibers in parallel, Applicants solved the challenge of stimulated and controlled growth by combining in the chip electrical and magnetic pulses and neurotrophic factors, and providing continuous communication with external monitors. The chip integrated circuit provides each TSV with a signal generator, field generator, power source, potential sensor, and transceiver, and is configured to apply and monitor electrical signals, currents, magnetic fields and potentials for each TSV. Specifically, the electronic signals may initiate a predetermined action potential in the adjacent nerve fiber end, and a sensor is configured for sensing the action potential in the nerve fiber end.

### BRIEF DESCRIPTION OF THE DRAWINGS

FIG. 1 shows a schematic top view of a semiconductor system integrated with through-silicon vias (TSV) for nerve regeneration according to an embodiment of the invention.

FIG. 2 displays a schematic cross section of the semiconductor system in FIG. 1, integrated with through-silicon vias (TSV) for nerve regeneration according to an embodiment of the invention.

FIG. 3 shows an enlargement of the portion marked in the schematic cross section of FIG. 2, detailing a plurality of layers of dielectric and conductive materials along the TSV.

FIG. 4 illustrates schematically an enlargement of another embodiment of the invention, showing an additional layer of iron with the plurality of layers of dielectric and conductive materials along the TSV. A plurality of solenoid windings, formed by the multi-level metallization of the integrated circuit, surround the TSV externally near the first chip surface.

FIG. 5 is an enlargement of the portion marked in FIG. 4.

FIG. 6 shows a schematic cross section through a semiconductor system, integrated with through-silicon vias (TSV) for nerve regeneration by nerve fiber growth according to the invention, with a neuron attached to the second chip surface opposite the first chip surface having the integrated circuit.

FIG. 7 illustrates schematically an example of a nerve fiber regrowth system based on semiconductor chips with the TSVs of the invention.

### DETAILED DESCRIPTION OF THE PREFERRED EMBODIMENTS

In order to successfully recover the use of severed limbs or organs, or to restore sensation or movement to a tissue, or to add biological functions to artificial machines such as prosthetic devices, it is first necessary to separate and functionally connect the severed nerve endings of the biological part. In some cases, neural extension outgrowth is promoted, in which nerve fibers, or axons, develop and extend into and through channels of an interface or linking device located between the severed nerve ends. Alternatively, for recording nerve impulses or implementing a bionic device, it is necessary to create an effective nerve interface with the opposing ends of a severed nerve. An effective or functional nerve interface and/or bridging device allows the reconnected neural circuit to be excited appropriately. In exemplary embodiments of the invention, a silicon chip manufacturing process similar to that employed for making semiconductor devices is used to generate an interface surface that is suitable for the separation and connection of nerve endings.

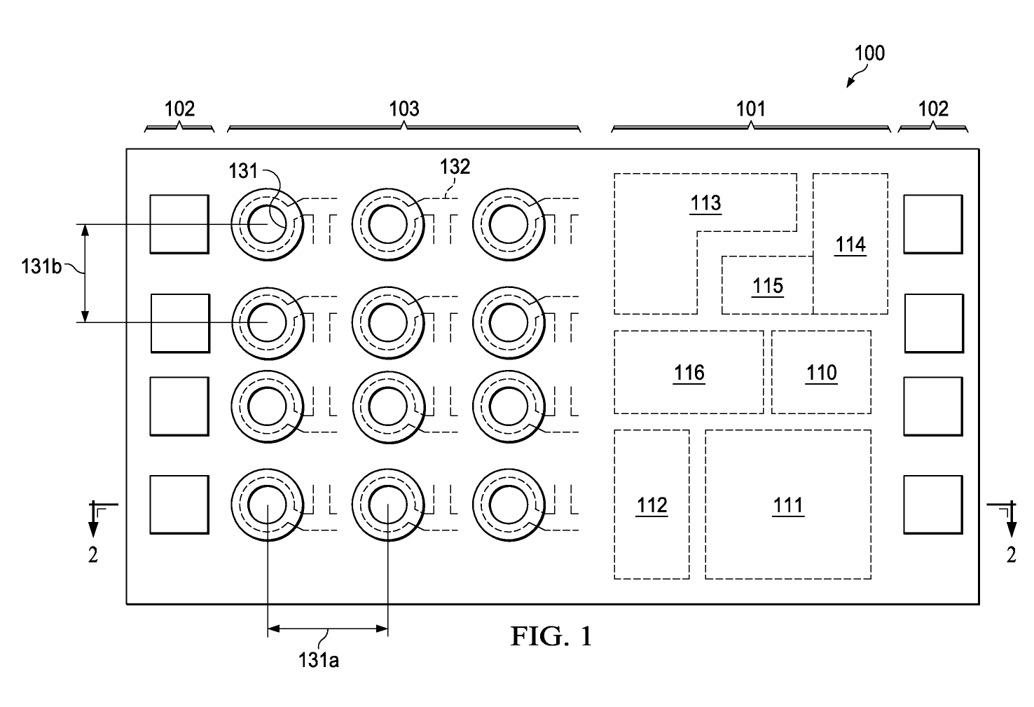

FIG. 1 illustrates the top surface of an exemplary embodiment of the invention. A semiconductor chip, generally designated 100, has a rectangular outline of 0.5 by 1.0 mm. The preferred semiconductor material is silicon; some embodiments, though, use silicon germanium, gallium arsenide, or any other semiconductor compound employed for batch production. Other embodiments may have chips of larger or smaller dimensions; the chips may be square shaped. The chip includes electronic circuitry displayed as an integrated circuit (IC) 101 with contact pads 102, and a plurality of through-silicon vias (TSV) 103. For the sake of descriptive clarity of the functionality, FIG. 1 shows these chip components as separate, distinct entities; in most actual embodiments, however, these components have an intermixed distribution in order to keep interconnection and routing simple and economical. Similarly, the elements of the IC are shown in FIG. 1 as separate entities, but they are preferably integrated in actual embodiments.

The elements of the exemplary integrated circuit 101 are shown in dashed outlines in FIG. 1; they include, but are not limited to, a power source 110, a memory 111, a microprocessor 112, a signal generator 113, a field generator 114, a potential sensor 115, and a transceiver 116. Working in an integrated fashion, these elements are configured to apply and monitor electrical signals, electrical current, magnetic fields, and potentials for each TSV. Transceiver 116 can be tuned for a system of radio frequency transmitter and receiver of nerve fiber (axon) growth. There may be more numerous functions in other embodiments.

The contact pads 102 may be adjusted to serve as pressure contacts, or as pads for wire ball bonds, or metal bumps, or solder bodies. The actual selection depends on the encapsulation or package of chip 100 to be compatible with implementation in specific organic body parts.

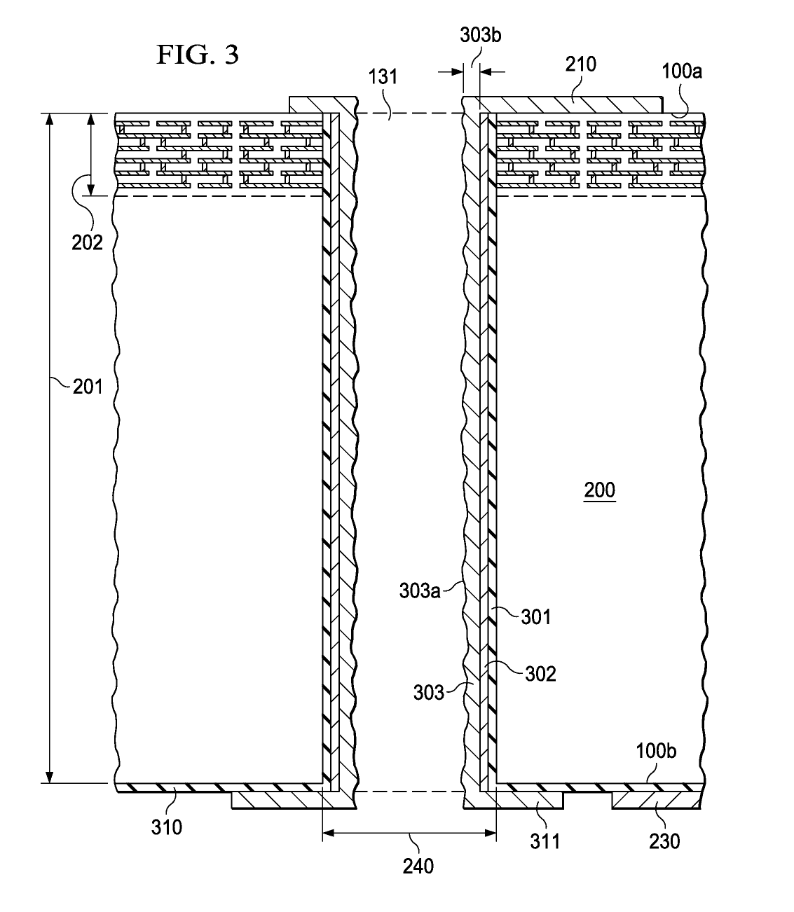

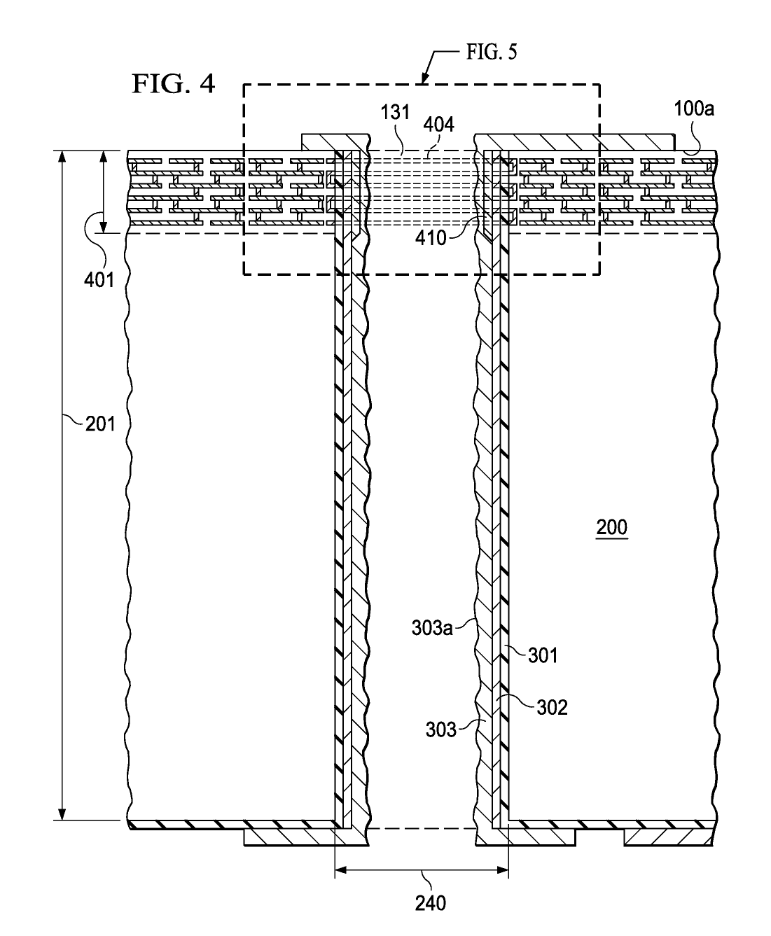

When the IC is processed through its numerous fabrication steps, the silicon material is still in wafer form and has a thickness between about 200 and 350 μm. Before the deposition step of the final metal layer, preferential etches open deep holes 131 with straight walls into the surface illustrated in FIG. 1. The selection of the etch solution depends on the crystalline orientation of the single-crystal silicon; appropriate choices can be found in the semiconductor literature. The process steps from the etched holes to the finished TSVs for this application are described below (see FIGS. 3 and 4). As the last process step, the final metal layer is deposited on the chip surface and patterned for electrically interconnecting each TSV with the circuitry 101. Portions of the metal connections 132 are schematically indicated in FIG. 1 by dashed lines.

The plurality of TSVs may be distributed across the chip area in any manner suitable for best functioning of the IC, or the plurality may be arrayed in a regular pattern as shown in FIG. 1, for best functional nerve regrowth. The regular pattern of the array may include rows of TSVs spaced at a pitch 131a center-to-center, and lines of TSVs spaced at a pitch 131b center-to-center. Pitches 131a and 131b may be different or identical. Preferred pitches range from about 25 to 50 μm center-to-center.

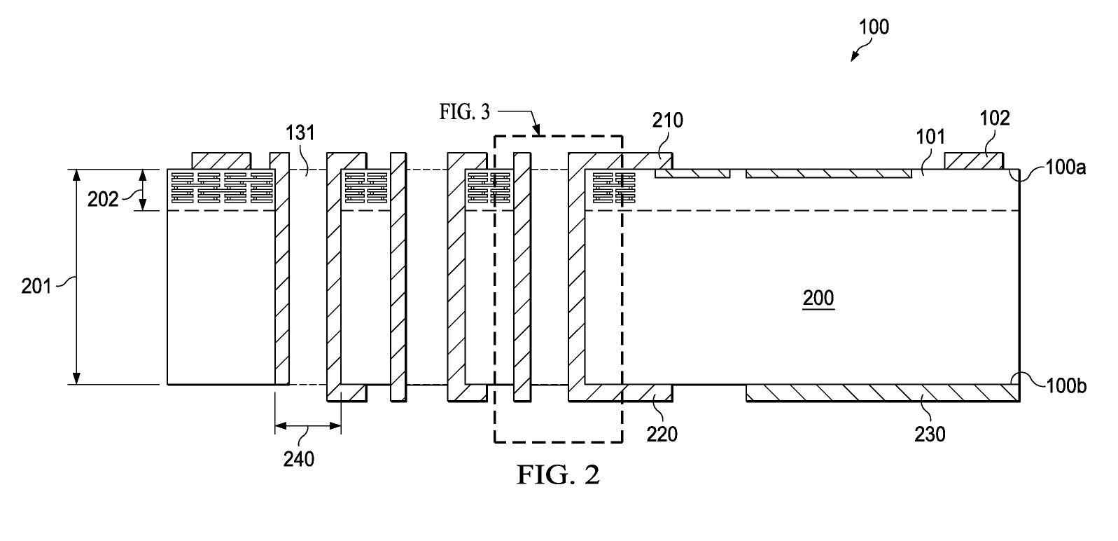

FIG. 1 includes phantom markers “2” to indicate the line along which a cross section is taken in order to create FIG. 2. The semiconductor chip 100 has a first surface 100a, a second surface 100b, and a thickness 201. In preferred embodiments, thickness 201 is in the range from about 70 to 150 μm (but may be thinner or thicker) and the depth 202 of the integrated circuit 101 at first surface 100a is between about 6 to 12 μm dependent on the number of metallization levels employed. FIG. 2 shows conductive connections 210 from the TSVs to the integrated circuit 101 on surface 100a; in other embodiments, these connections may be embedded as part of the circuitry. On the other hand, contact pads 102 are shown as part of the IC, but in some embodiments they may be added on surface 100a.

FIG. 2 further shows attachment pads 220 for nerve ends and nerve fibers (axons), and attachment pads 230 for neurons. These schematic displays should not be understood in a limiting sense, but rather as examples for a wide spectrum of specific arrangements. Dependent on the medical application, the preferred metal for connections 210 and pads 220 and 230 may be selected from a group including iridium, gold, platinum, palladium, and silver.

As FIG. 2 illustrates, the through-semiconductor via-holes 131 extend from the first surface 100a through the chip thickness 201 to the second surface 100b. The TSVs are parallel to each other. At the semiconductor material 200, the side walls of each TSV are straight, but not necessarily parallel to each other; preferably, the TSV has a cylindrical shape, but may in some embodiments have the shape of a truncated cone. In other embodiments, the cross section of holes 131 may be rectangular, hexagonal, or in any other outline compatible with the crystalline orientation of the semiconductor material. The preferred diameter 240 of the etched TSV ranges from about 10 to 40 μm, but may be considerably smaller or larger. The final diameter depends on the number and thickness of the metal layers inside the hole (see FIG. 3).

FIG. 2 includes phantom lines to indicate the portion, which is enlarged in FIG. 3 in order to illustrate the detail of an exemplary TSV according to the invention. The exemplary TSV 131 extends through the chip thickness 201, including the thickness 202 of the integrated circuit, and has a uniform diameter, designated 240 after the etching step. As stated above, this hole of about 70 to 150 μm depth is etched while the semiconductor material 200 is still in wafer form. In the preferred process flow, a dielectric compound such as silicon nitride or silicon dioxide is deposited on the TSV sidewalls after the etching step in order to create a thin (<1 μm) insulating layer 301 between the semiconductor material 200 and the conductive layers inside the TSV.

Thereafter, a metal seed layer 302 is deposited (<1 μm thick) on the insulating layer 301. The selection of the seed metal or metal compound depends on the choice of the metal layer 303. The deposition of the thicker metal layer 303 (preferable thickness 303b between about 1 and 5 μm, for some applications thicker) may be performed before the wafer thinning (grinding) process or after the thinning step. In either variation, it is preferred that the inner surface of layer 303 is rough, as indicated in FIG. 3 by contour 303a, in order to provide mechanical gripping and support for the nerve fiber to grow inside the TSV. If layer 303 is deposited before wafer grinding, the metal deposition on surface 100a for the TSV connection 210 to the IC may preferably be performed concurrently with the deposition inside the hole.

The process step of thinning the wafer, by grinding or etching or both, continues until the bottom of the via hole is exposed and the TSV is opened at the second surface 100b; as mentioned, the remaining semiconductor thickness 201 is preferably between about 70 and 150 μm. After the thinning step, an insulating layer 310 may be deposited on second surface 100b, for example by using a polyimide compound, followed by patterned metal connection 311, which may, for instance, be made of eutectic gold-germanium alloy (12.5 weight % Ge, eutectic temperature 361° C.) and provides an electrical contact to TSV layer 303 from the second surface 100b. At the same time, the nerve attachment pad 230 is created.

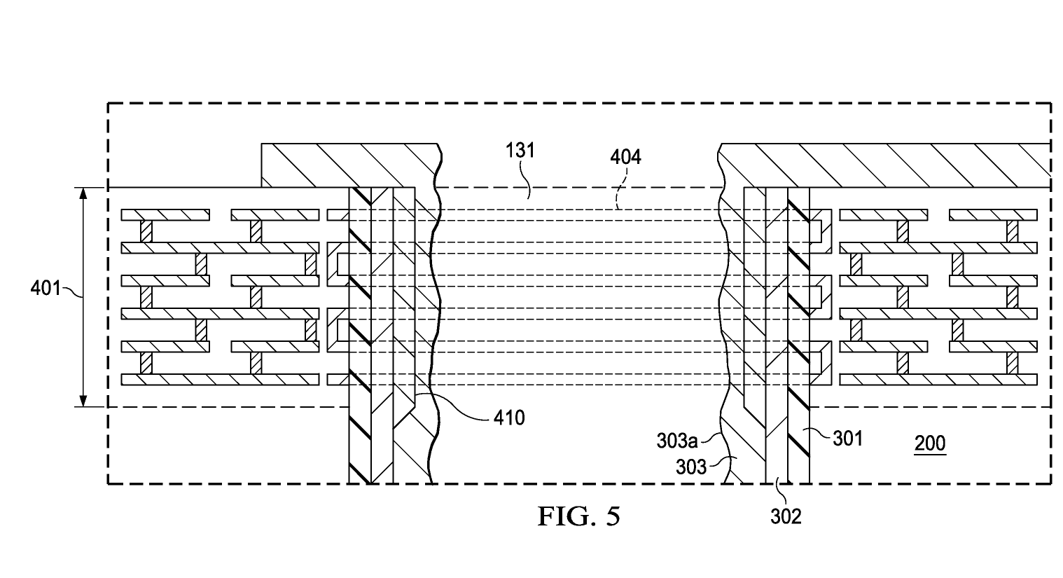

Another variation of the TSV metallization is illustrated by the embodiment in FIG. 4 and enlarged in FIG. 5. The integrated circuit (IC), built into the first surface 100a of semiconductor material 200, extends through a thickness 401 from first surface 100a. Included in the IC thickness are, in the example of FIG. 4, six levels of metallization, interconnected by a multitude of metal-filled vias (in other embodiments may be fewer or more metallization levels). The metal levels are patterned so that on each level a winding 402 around the opening of TSV 131 is formed, which continues into a via connection 403 to the respective winding formed by the next metallization level. The via connections of the sequential windings are placed so that an electric current can progress from one winding to the next while flowing in the same clock direction. In this fashion, a plurality of interconnected windings are formed (total of six windings in FIG. 4), which are insulated from each other and constitute a solenoid 404 surrounding the opening of TSV 131. A current flowing through solenoid 404 in a specific continuous clock direction creates a magnetic field inside the TSV for the length 401 of the solenoid with the magnetic field strength being approximately uniform; the magnetic field lines inside the TSV are approximately parallel to the TSV length 201 and exit the solenoid openings to close outside the solenoid.

Further shown in FIG. 4 (and enlarged in FIG. 5) is an iron layer 410. As mentioned, after the via-hole with diameter 240 has been opened, the insulating layer 301 and the seed layer 302 are deposited on the sidewalls of the hole. After these deposition steps, it is advantageous for some embodiments to deposit a layer 410 of iron on the seed layer so that iron layer 410 extends approximately the length of the solenoid (IC thickness 401), forming an iron mantel inside of, yet isolated from the solenoid. A preferred iron layer thickness is between about 5 and 15 μm. The iron mantel acts to amplify the magnetic field (due to iron's high magnetic permeability) created by the current through the solenoid 404 and to concentrate the field strength inside the iron layer thickness. At the end of the iron mantel, the magnetic field has a locally high field strength. This relatively intense magnetic field, especially when pulsed, may influence the growth of nerve fibers.

In order to minimize the cost of the discrete devices 100 as discussed in FIGS. 1 to 4, it is preferred to batch-process a plurality of these devices in semiconductor wafer form, including fabricating the IC on the first wafer surface, creating the plurality of TSVs through the wafer thickness, and back-grinding the second wafer surface to the final wafer thinness. After the thinning step, the wafer may receive the patterned metallization described in FIG. 2, while the first surface is protected (for example by photoresist or by a temporary carrier). The discrete devices 100 are then singulated from the wafer; a number of techniques such as sawing with a rotating blade saw, laser beam, water jet, are available. The discrete unit 100 may be electrically connected by pressure contacts, or by bonding with wires or metal bumps, dependent on the intended method of affixing neurons for nerve regeneration and implanting of the device into body tissues.

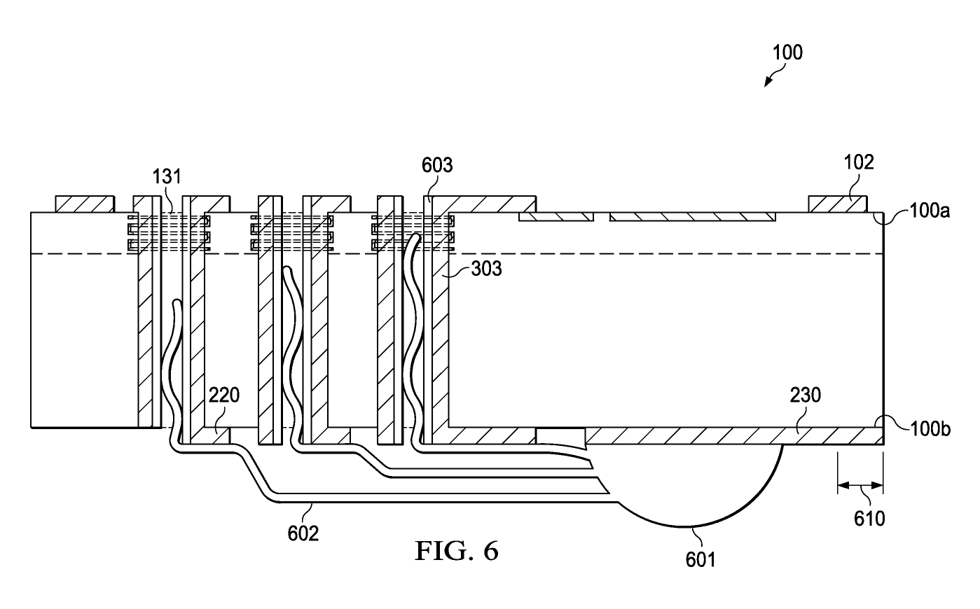

FIG. 6 illustrates schematically a neuron 601 placed on attachment pad 230 on the second surface 100b of chip 100; neural adhesive solutions may be used. If needed, the placement may leave some area of width 610 available for an optional electrical (pressure) contact to pad 230 so that pad 230 can be coupled to external power sources and therewith to the circuitry on first chip surface 100a. Neuron 601 has a plurality of nerve fibers (axons) 602 of a typical diameter of about 10 μm. In order to promote nerve growth and axon extension through the TSV, each axon 602 is aligned with a respective hole 131 of a TSV and may also be placed on an attachment pad 220. Pads 220 can be electrically biased and pulsed; they are involved in receiving and emitting electrical potentials, and collecting and recording action potentials of nerve ends. Assisting in monitoring the nerve fiber growth may be the transceiver 116, which is incorporated in the chip circuitry and tuned for a system of radio frequency identification. Furthermore, attachment pad 230 can be biased and pulsed, since it may be electrically connected by contact pad 610 to external circuits and the circuitry on first chip surface 100a. As stated above, metal sidewalls 303 of the TSV are connected to the circuitry on first chip surface 100a and contact pads 102 serve the connection to external parts and power supplies, and therewith to the contact pads on second chip surface 100b.

FIG. 6 further shows layers 603 of biological neurotrophic growth factors (for example growth-enhancing protein or peptide molecules, bone-derived neurotrophic factors, neurotrophin-1, neurotrophin-3, and neurotrophin-4) coated onto the surface metallization 303 for stimulating the growth of the axons. A preferred coating method involves dipping or bathing chip 100 in a growth-promoting solution and rinsing off surfaces 100a and 100b, whereby the biological substance is left in the vias or on the sidewalls. Further suitable polymers can be found in the literature and in patents; for instance, see A. Mensinger et al., “Chronic Recording of Regenerating VIIIth Nerve Axons . . . ”, J. Neurophysiology, vol. 83, pp. 611-615, 2000; N. Syed, “Method and Apparatus for Guiding Growth of Neurons”, Internat. Publ. #WO 2007/009235; J. Flaherty, “Nerve Regeneration System and Lead Devices Associated therewith”, Internat. Publ. #WO 2008/005843. The growth factors may also be transported to through micro-fluidic channels. Additionally, Nanoparticles, such as nanobeads and quantum dots, may also be used to provide growth enhancement and/or as markers. They may also be immobilized at specific sites on the surface of the substrate or in the vias, or delivered to the neuronal milieu through their un-caging'-via electric fields or through micro-fluidic channels. These nano-beads and quantum dots may be pre-labeled with various markers (such as fluorescent markers, or biological markers) for highly selective neuronal labeling, or may be designed to deliver various protein molecules and gene perturbation molecules.

In the configuration of FIG. 6, chip 100 can satisfy a number of functions for activating and monitoring growth and regeneration of neuron 601. These functions include: To emit and sense electrical currents; to emit and sense electrical pulses and electrical fields; to sense nerve action potentials; to generate and sense magnetic fields; to receive, collect, and record neural activities; to facilitate connection of severed axons to bionic devices; to control bionic devices. Included in the structure of device 100 in FIG. 6 is the feature to increase the electrical current through a TSV sidewall to such high level that the growth of axons is disrupted for instance by searing. Since the action potential is the transient change in electrical potential at the surface of a nerve cell occurring at the moment of excitation, the IC of the chip 100 in FIG. 6 includes components to create, sense, and monitor action potentials especially at the tip of growing axons.

Another embodiment of the invention is a method for enhancing nerve regeneration by using an apparatus 100 as described in FIGS. 1 to 4. In the first process step, at least one neurotrophic factor may be supplied to each TSV. Next, a severed nerve end or axon is secured to a discrete device 100, as illustrated in exemplary fashion in FIG. 6. Pads and contacts for holding the nerve ends are located on the second chip surface opposite the integrated circuit on the first chip surface. The severed axon is then guided into a respective TSV, where it touches the metallic sidewall on its tip or on some area of its length.

In the next process step, an electrical signal or pulse is applied to the metallic side wall of the TSV, exciting the portion of the axon touching the sidewall. The pulse results in a transient change in electrical potential at the surface of the axon, i.e. the pulse results in a nerve action potential. In feedback, this action potential is monitored by the IC. The electrical pulses are configured to stimulate directional extension of axons into the TSV and along the TSV through the thickness 201 of the chip. The process sequence can be repeated numerous times at consecutive time intervals, and over a long period of time. It thus lends itself to stimulate axon growth as well as monitoring the growth process.

In additional process steps, electrical pulses can be applied through the solenoid around the TSV at the first chip surface, creating pulses of magnetic field. It can be observed how these magnetic filed pulses may influence the axon growth in the TSV, and to what extent pulsed or continuous magnetic fields affect nerve ion channels.

Based on monitoring of the action potentials, the electrical signals, or pulses, as well as the magnetic field pulses can be modified so that a controlled feedback loop can be established between the pulses, the action potentials, and the fields. In this manner, the axon growth through a plurality of TSVs can be approximately equalized so that a plurality of axons, re-growing in parallel as an array, would arrive approximately at the same time at the first chip surface. The growth of an axon, which would not follow this expected growth as an array, could be disrupted by increasing the electrical current through the respective TSV to the level of searing the axon.

As mentioned above, it is advantageous to preserve the semiconductor chip 100 around the newly grown axons, in particular since the semiconductor material of chip 100 offers itself as protective sheaths of the neuron extensions. Further, the magnetic field generated by the IC for each axon can be used as a long-term research vehicle to study the nerve ion channels as a function of the magnetic field strength.

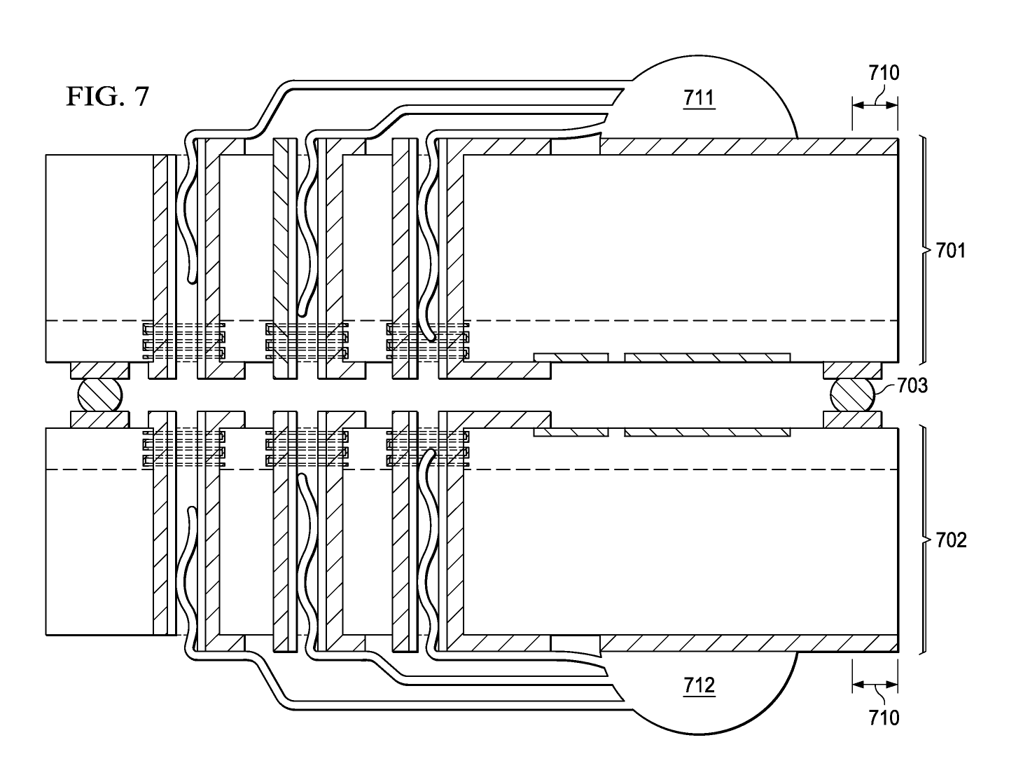

After sufficient axonal regrowth into and through the TSV, the nerves containing the regrown axons, once established, will potentially be able to make good electrical contact with a target tissue to restore sensation or mobility, or to allow a bionic device or prosthesis to function as designed. An exemplary system is schematically illustrated in FIG. 7. Two analogous semiconductor chips 701 and 702 with a plurality of TSVs are coupled together in flip-chip fashion by conductive polymer bodies 703. The chips are a mirror image of each other to allow precise aligning of the respective TSVs. For some applications, the polymer material of bodies 703 may be a thermoplastic compound; for other applications, the polymer material may be a B-staged thermoset compound with a low polymerization temperature. Each chip has a nerve cell (711 and 712, respectively) attached. The severed axons of the cells are guided into the respective unidirectional and parallel TSVs for regrowth, until they finally meet. To facilitate and insure the meeting of the nerve ends, it may be advantageous to keep the size of coupling bodies 703 small, in some applications so small that chips 701 and 702 almost touch. As mentioned above, the axon growth may be monitored by the antenna incorporated in each chip circuitry and tuned for radio frequency identification. The semiconductor material of the chips remains as protective sheath for the sensitive axons. Pads 710 serve as the contacts to connect the system to external parts (such as power supply and monitor), in some application by pressure contact, in other applications by affixing conductive connections.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. As an example, the invention applies to any semiconductor material for the chips with TSVs, including silicon, silicon germanium, gallium arsenide, or any other semiconductor or compound material used in manufacturing.

As another example, in a plurality of TSVs, the diameter of the TSVs may be uniform or it may be different from each other. The TSVs may be arrayed in an orderly pattern, or randomly. The TSVs sidewalls may have one or more metal layers. The innermost layer may have a smooth surface or a rough surface.

As another example, the semiconductor chip may be free of an encapsulation, or it may be in an additional package. The system may have an electrically connective ribbon, or it may include a battery.

It is therefore intended that the appended claims encompass any such modifications or embodiments.

What is claimed is:

1. An apparatus for enhancing stimulation, regeneration, and control of nerves, said apparatus comprising:
a semiconductor chip having a thickness, a first surface including electronic circuitry, and a second surface including attachment pads for nerve ends;
a plurality of through-semiconductor via-holes (TSVs) extending from the first surface through the chip thickness to the second surface, the via having an electrically conductive inner side wall including conductive external connections to the circuitry and to the nerve attachment pads; and
the electronic circuitry including an integrated circuit coupled to a signal generator, a field generator, a power source, a potential sensor, and a transceiver, and configured to apply and monitor electrical signals, currents, magnetic fields, and potentials for each via.

2. The apparatus of claim 1 wherein the vias are parallel to each other.

3. The apparatus of claim 2 further including certain vias of the plurality arrayed in a regular pattern.

4. The apparatus of claim 3 wherein the regular pattern includes rows and lines of vias spaced at a pitch center-to-center.

5. The apparatus of claim 4 wherein the vias have a diameter between about 10 and 40 μm and a depth between about 70 and 150 μm.

6. The apparatus of claim 5 wherein the pitch center-to-center is between 25 and 50 μm.

7. The apparatus of claim 6 wherein the electrically conductive via side wall includes a stack of layers comprising an innermost metal layer selected from a group including gold, platinum, iridium, palladium, and silver, contiguous with a seed layer, contiguous with an outermost insulating layer on the semiconductor material.

8. The apparatus of claim 7 wherein the innermost metal layer has a roughness suitable to mechanically support axon growth.

9. The apparatus of claim 8 further including solenoid windings externally surrounding the via near the first surface.

10. The apparatus of claim 9 wherein the number of solenoid windings equals the number of metallization levels of the integrated circuit.

11. The apparatus of claim 10 wherein a portion of the electrically conductive via side wall further includes a layer of iron sandwiched between the metal layer and the seed layer.

12. The apparatus of claim 1 wherein the electrical signals are configured to initiate action potentials in the nerve ends.

13. The apparatus of claim 1 wherein the electronic circuitry includes sensors for action potentials in nerve ends, the sensors coupled to the integrated circuit.

14. The apparatus of claim 1 wherein the electrical current may have a magnitude to disrupt axon growth through the via.

15. The apparatus of claim 1 wherein the vias contain at least one neurotrophic factor.

16. The apparatus of claim 1 wherein the semiconductor material between the vias provides the protective sheath for axons growing inside the vias parallel to each other.

17. The apparatus of claim 1 further including a transceiver tuned for a system of radio frequency identification of nerve fiber growth.

18. A method for enhancing nerve regeneration, comprising:
securing a severed nerve end to a nerve end pad located on the second chip surface of the apparatus of claim 1;
guiding an axon extension into the respective via;
applying an electrical signal to the nerve end, thereby initiating an action potential in the nerve end; and
monitoring the potential in the nerve end at consecutive time intervals during the axon growth.

19. The method of claim 18 further including, after applying, generating a magnetic field inside the via near the first chip surface for affecting nerve ion channels.

20. The method of claim 19 further including, after monitoring, modifying the electrical signal and the magnetic field, causing a controlled feedback loop between signal, action potential, and field.

21. The method of claim 20 further including, after modifying, providing an electrical current to the via side wall to disrupt the axon growth.

22. The method of claim 18 further including, before securing, supplying at least one neurotrophic factor to each via.

23. The method of claim 18 further including, after monitoring, applying an electric field gradient to stimulate the directional growth of the axons along the vias.

24. The method of claim 18 further including monitoring the nerve ion channels as a function of the magnetic field strength.

25. The method of claim 18 further including, after monitoring, preserving the semiconductor material of the chip as protective sheaths around the newly grown axons as neuron extensions.
